(19) United States
(12) Patent Application Publication
(10) Pub. No.: US 2014/0074049 A1
(43) Pub. Date: Mar. 13, 2014

(54) Catheter Connector for Measurement of Physiological Signals

(71) Applicant: AngioDynamics, Inc., Latham, NY (US)

(72) Inventors: Franciscus Ludovicus Maria Johannes Veldhuijzen, Liessel (NL); Lambertus Wilhelmus Johannes Elberse, Oss (NL)

(73) Assignee: AngioDynamics, Inc., Latham, NY (US)

(21) Appl. No.: 14/022,379

(22) Filed: Sep. 10, 2013

(30) Foreign Application Priority Data
Sep. 11, 2012 (NL) 2009448

(51) Int. Cl. A61M 39/10; A61M 39/26; A61B 5/042

(52) U.S. Cl. CPC A61M 39/10; A61B 5/042; A61M 39/26
USPC 604/256

(57) ABSTRACT

The present invention relates to a connector, for use with a catheter provided with a catheter valve, comprising a liquid channel, for transporting a liquid from a first to a second end of the connector, or vice versa, a first coupler, provided on the first end of the connector, to couple the connector and the catheter, a second coupler, provided on the second end of the connector, to couple the connector and a liquid administering—and/or measuring device, a pin-shaped part, from the first end of the connector extending in substantially axial direction, to open the catheter valve when the connector is coupled to the catheter.

The present invention relates to a connector, for use with a catheter provided with a catheter valve and a device to measure intravascular physiological signals.

To administer medication to patients in hospitals, doctors often make use of drips, wherein a hollow needle is passed through the skin of a patient, directly into the vein. Though the hollow needle a watery solution can be administered to the patient through slow intravenous of hypodermal infusion. Drawback of the use of a hollow needle is that such a needle can only be used up to a few days alter application. Therefore patients, who need prolonged administration of drugs, have to undergo multiple applications of hollow needles, which is a burden on the patients, and required tremendous time on personal.

To overcome this drawback, people who need prolonged medication are often provided with PICC's, so-called peripherally inserted central catheters. These catheters are inserted through the vasculature of the patient and positioned such, that the tip of the catheter debouches on a location where the administered medication can be diluted and transported quickly. A location often used to place the tip of the catheter is the transition between the right atrium and the vena cava. To localize the tip of these catheters use is made of X-ray radiation, to detect and guide the tip to the proper location. Use of X-ray is a costly and burdensome for the patient.

Tip localization of these catheters can also be done without the use of harmful radiation when the catheters are provided with ECG sensors. As the position of the catheter in the vasculature of the patient changes, so does the ECG signal measured by the ECG sensor on the catheter. This technique is known; US 2008/0097232 for instance discloses a method of locating the on of a central venous catheter, using ECG waveforms and P-waves.

Tip localization using ECG signals depends on the interpretation of the P wave and the of the ECG signal, wherein the P wave is often used as a trigger, in great detail. When the tip of the catheter approaches the sine node of the heart, which lies close to the transition between the right atrium and the vena cava, the shape of the P wave changes.

Based on real-time measurements of the ECG a person skilled in the art is therefor able to determine whether the catheter has to be pushed in further, is in the optimal position, or should be retracted. These determinations could also be automated and be sent to a mobile device, such as a mobile phone, which will only display directions to proceed, stop, or retract.

New generation PICC's however are provided with a proximal valve, which prevents the flow of liquids through the catheter and separates the part of the catheter that enters the patient from the part that is accessible to administer medication. This valve renders it impossible to measure ECG signals with an ECG sensor located on the tip of the catheter and to send these signals to a measuring device located outside the patient, since these signals cannot traverse over the valve. The tip localization method using an ECG sensor can therefor not be used when these newer PICC's applied to patients.

It is therefor the goal of the present invention to overcome these drawbacks to be able to measure ECG signals suitable for catheter localization when use is made of a catheter with a proximal valve.

The invention thereto proposes a connector for use with a catheter provided with a catheter valve, comprising a liquid channel, for transporting a liquid from a first to a second end of the connector or vice versa; a first coupler, provided on the first end of the connector, to couple the connector and the catheter; a second coupler, provided on the second end of the connector, to couple the connecter and a liquid administering—and/or measuring device and a pin-shaped part, from the first end of the connector extending in substantially axial direction, to open the catheter valve when the connector is coupled to the catheter.

When the connector is coupled to a catheter provided with a catheter valve, the pin-shaped part extends beyond the catheter valve, creating a fluid passage, and thus in case of a fluid being present, a conductive connection over the catheter valve, whereas the coupling ensure a liquid tight connection between the catheter and the connector. The liquid tight connection between the different parts can be made by using, for instance, rotary couplings.

The connector according to the invention allows performing of electric measurements when using a catheter valve, and more particular, performing electric measurements when placing the catheter, which was, according to the state of the art, hindered, if not made not possible via the electric properties of a liquid in the valve. Such liquid may for instance comprise a saline solution.

In an alternative embodiment, the invention proposes a connector, for use with a catheter provided with a catheter valve, comprising a first coupler, provided on the first end of the connector, to couple the connector and the catheter an at least partly electrically conductive pin-shaped pan, from the first end of the connector extending in substantially axial direction, to open the catheter valve when the connector is coupled to the catheter an electric conductor, conductively coupled to the pin-shaped part, which extends from the channel to an outside of the channel, to connect a measuring device.

Such connector can be used during the placement of the catheter, and has to be exchanged with a syringe for administering fluids to the vascular system of a patient.

In an embodiment of the present invention the pin-shaped part is hollow. The hollow pin-shaped part creates a liquid channel that extends from the second end of the connector to the end of the pin-shaped part, which lies beyond the catheter valve when the catheter is connected to the connector.

In an embodiment of the present invention the pin-shaped part is for example between 5 and 50 mm long, and has a diameter between 0.5 and 2.5 mm. The sizes (lumen & Length) of the pin depends of the model and size of the used catheter. The length of the pin-shaped part depends mainly on the distance between the coupling means of the catheter and the position of the catheter valve. The diameter of the pin-shaped part depends mainly on the diameter of the catheter, as well as on the diameter of wire that has to be able to be transported through the catheter in operating procedures.

In another embodiment the connector comprises an electric conductor; which extends from the channel to an outside of the channel, to connect to a measuring device. The electric conductor enables the connecting of a measuring device such as an electrocardiograph to the connector, to relay physiological signals received by the tip of the catheter to the measuring device. De electric conductor may be a conductive strip, which extends outside the connector, where the signals can be transported to the measuring device through simple electric wiring. The measuring device can also be used to transmit a signal to the connector when a measuring method requires to do so.

The invention also related to a combination of a connector and a liquid administering device, such as a syringe. In that embodiment the channel extends through inside the syringe, and the syringe could be equipped with an electric conductor, which extends from the channel to an outside of the channel, to connect to a measuring device, such as a electrocardiograph.

The invention also relates to a device to measure intravascular physiological signals, comprising a peripherally inserted central catheter, comprising a tip, at a first side of the catheter, to insert the catheter in the vasculature and to receive physiological signals; a valve, at a second side of the catheter, to regulate access to the tip; at least one lumen, to connect the tip and the valve, and to transport the physiological signals; a measuring device, such as a electrocardiograph, to measure the physiological signals characterised by a connector for a conductive connection between the tip of the catheter and the measuring device.

The combination of all components, being the catheter, the connector and the measuring device, can be used to measure and interpret intravascular received physiological signals, which are transported through the catheter valve by using the connector, to be transported towards a measuring device such as a electrocardiograph, for measuring and interpreting the received signals.

The invention will now be elucidated into more detail with reference to the following figures, wherein;

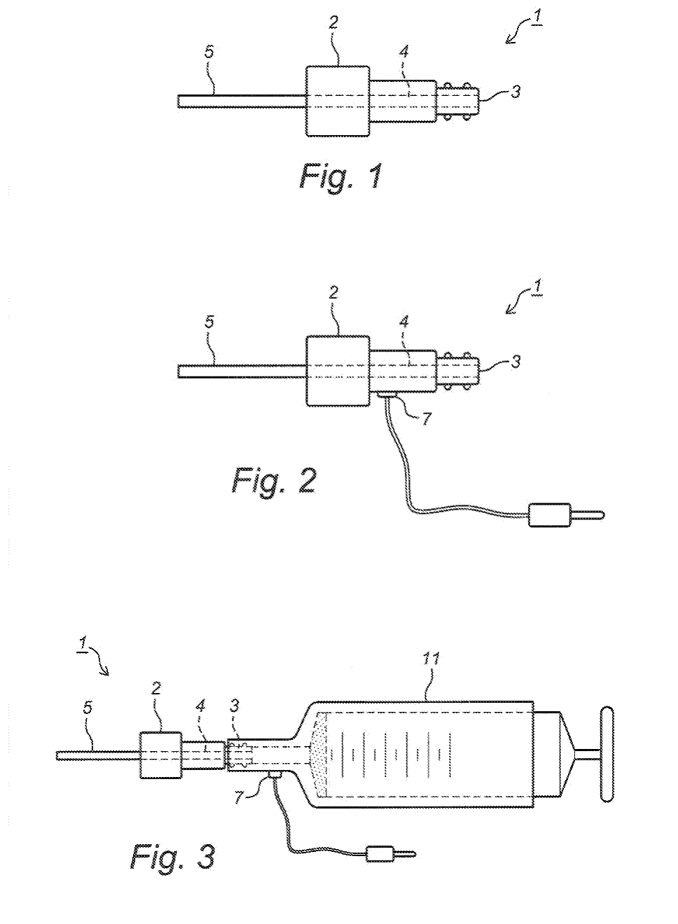

FIG. 1 shows a connector according to the present invention

FIG. 2 shows a connector provided with an electric conductor

FIG. 3 shows a combination of a connector and a syringe provided with an electric conductor

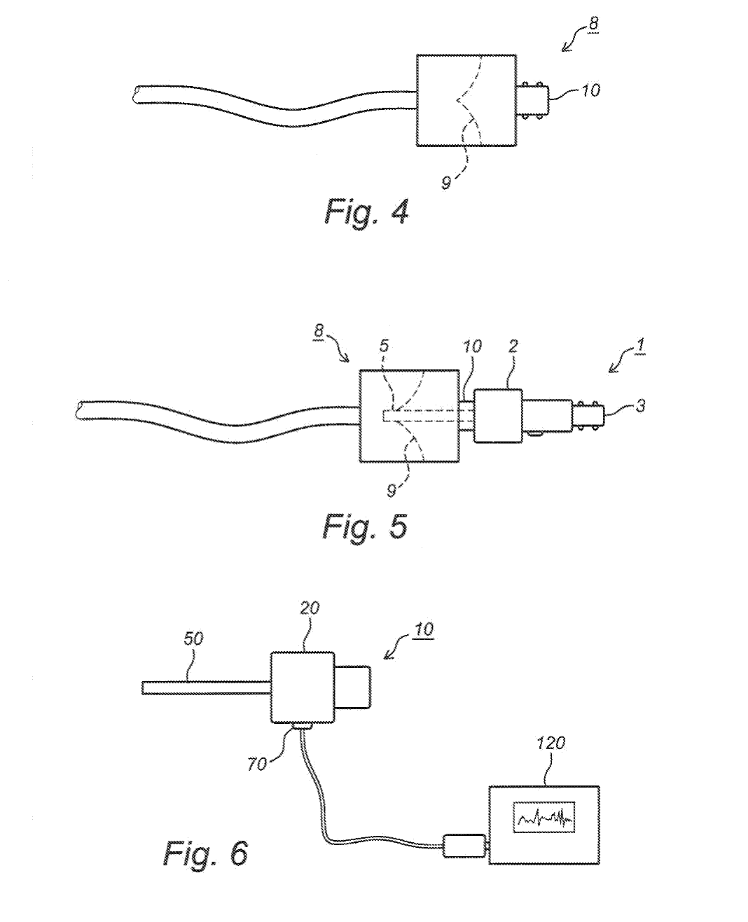

FIG. 4 shows a catheter, connectable to a connector according to the present invention

FIG. 5 shows a catheter, coupled to a connector.

FIG. 6 discloses a different embodiment of the invention.

FIG. 1 shows a connector 1, comprising a first coupler 2 to couple the connector and the catheter, a second coupler 3 to couple the connector and a liquid administering—and/or measuring device, liquid channel 4 for transporting a liquid from a first to a second end of the connector 1 and pin-shaped part 5 to open the catheter valve when the connector is coupled to the catheter.

FIG. 2 shows a connector 1, first and second couplers 2 and 3, liquid channel 4, pin shaped part 5, wherein the connector is provided with an electric conductor 7 which extends from the channel loan outside of the channel to transport received signals from the tip of a catheter to a (not shown) measuring device. The transport of signals through the liquid channel is preferable performed via a conductive solution, such as physiological salt or blood.

FIG. 3 shows a connector 1, first and second couplers 2 and 3, channel 4 and pin-shaped part 5, wherein the connector 1 is coupled to a syringe 11 through the second coupler 3. Syringe 11 is provided with an electric conductor 7, which extends from the channel to an outside of the channel, to transport received signals from the tip of the catheter to a (not shown) measuring device. The liquid channel 4 here extends till inside the syringe 11.

FIG. 4 shows a catheter 8, provided with a catheter (proximal) valve 9. The catheter is provided with coupling means 10, to couple the catheter 8 and the first coupler 2 of the connector 1.

FIG. 5 shows a connector 1, which is coupled to the coupling means 10 of the catheter 8 via the first coupler 2 of the connector 1. The pin-shaped part 5 of the connector 1 extends beyond the proximal catheter valve 9, which allows a conductive connection between the tip of the catheter and the second coupler 3 of the connector 1, by for instance injection of a conductive medium such as physiological salt, especially when the pin-shaped part 5 is hollow. The moment the pin-shaped part 5 opens the catheter valve 9 preferable coincides with the coupling of the catheter to the connector 1, such that the patient's blood can be guided through the connector and the liquid channel.

FIG. 6 shows a connector 10, for use with a catheter provided with a catheter valve, comprising a first coupler 20, provided on the first end of the connector 10, to couple the connector 10 and the catheter; an at least partly electrically conductive pin-shaped part 50, from the first end of the connector extending in substantially axial direction, to open the catheter valve when the connector is coupled to the catheter and an electric conductor 70, conductively coupled to the pin-shaped part 50, which extends from the connector 10, to connect a measuring device 120.

1. Connector, for use with a catheter provided with a catheter valve, the connector comprising:
a liquid channel, for transporting a liquid from a first to a second end of the connector;
a first coupler, provided on the first end of the connector, to couple the connector and the catheter;
a second coupler, provided on the second end of the connector, to couple the connector and a liquid administering and/or measuring device;
a pin-shaped part, from the first end of the connector extending in substantially axial direction, to open the catheter valve when the connector is coupled to the catheter.

2. The connector according to claim 1, wherein the pin-shaped part is hollow.

3. The connector according to claim 1, wherein the pin-shaped part extends beyond the catheter valve when the connector is coupled to the catheter.

4. The connector according to claim 1, wherein one of the first and second coupler comprise rotary couplings.

5. The connector according to claim 1, wherein the pin-shaped part has a length between 5 and 50 mm, and a diameter between 0.5 and 2.5 mm.

6. The connector according to claim 1, further comprising an electric conductor which extends from the channel to an outside of the channel, to connect a measuring device.

7. The connector according to claim 1, connected to and in fluid communication with a liquid administering device.

8. The liquid administering device of claim 7 further comprising an electric conductor, which extends from the channel to an outside of the channel, to connect a measuring device.

9. A connector for use with a catheter provided with a catheter valve, the connector comprising:
a first coupler, provided on the first end of the connector, to couple the connector and the catheter;
an at least partly electrically conductive pin-shaped part, from the first end of the connector extending in substantially axial direction, to open the catheter valve when the connector is coupled to the catheter;
an electric conductor, conductively coupled to the pin-shaped part, which extends from the connector, to connect a measuring device.

10. The connector according to claim 9, also comprising a measuring device, such as a electrocardiograph.

11. (canceled)
